(19) United States
(12) Patent Application Publication
(10) Pub. No.: US 2025/0032734 A1
(43) Pub. Date: Jan. 30, 2025

(54) CONTROL SCHEMES FOR MECHANICAL COUGH

(71) Applicant: Ventec Life Systems, INC, Bethell, WA (US)

(72) Inventors: Joseph Cipollone, Bothell, WA (US); Samir Saleh Ahmad, San Diego, CA (US)

(21) Appl. No.: 18/227,436

(22) Filed: Jul. 28, 2023

(51) Int. Cl. A61M 16/00

(52) U.S. Cl. CPC A61M 16/0054; A61M 16/024; A61M 2016/0027; A61M 2016/003

(57) ABSTRACT

This disclosure relates to control schemes for a system including mechanical cough functionality, and corresponding methods. Among other benefits, this disclosure reduces if not eliminates retrograde displacement of secretions within a patient's airway during a mechanical cough mode.

### TECHNICAL FIELD

This disclosure relates to control schemes for a system including mechanical cough functionality, and corresponding methods.

### BACKGROUND

Respiratory ventilation may be characterized as including both an inspiratory phase and an exhalation phase. During the inspiratory phase, inspiratory gases are drawn into the lungs, and during the exhalation phase, exhalation gases are expelled from the lungs.

Mechanical ventilators are used to assist with breathing. Conventional ventilators typically push inspiratory gases including oxygen into the patient's lungs. Many patients who use a ventilator also need other types of assistance related to treating and maintaining their airways and lungs. For example, some patients may use a nebulizer to deliver drugs to their lungs and/or airways. Further, some patients may need help clearing secretions from their lungs and/or airways.

Some patients may also need cough assistance. To use some known cough assistance devices, which may be referred to as mechanical insufflation-exsufflation (MIE) devices, a patient must be disconnected from mechanical ventilation, and connected to a separate device. After a cough assistance, or MIE, maneuver is performed, the patient must be disconnected from the MIE device, and reconnected to the mechanical ventilation. Often, suctioning of the patient airway is also performed after the patient has been disconnected from the MIE device and reconnected to the mechanical ventilation to remove secretions not adequately cleared from the patient airway during the MIE maneuver.

### SUMMARY

In some aspects, the techniques described herein relate to a system, including: a respiratory device configured to deliver fluid to a patient, wherein the respiratory device is operable in a mechanical cough mode; and a controller configured to issue one or more commands to the respiratory device such that, during an insufflation phase of the mechanical cough mode, (i) a flow rate of the fluid conducted to the patient by the respiratory device is substantially constant throughout the insufflation phase, or (ii) a flow rate of the fluid conducted to the patient by the respiratory device gradually increases throughout the insufflation phase.

In some aspects, the techniques described herein relate to a system, wherein, during the insufflation phase of the mechanical cough mode, a flow rate of the fluid conducted to the patient by the respiratory device is substantially constant throughout the insufflation phase.

In some aspects, the techniques described herein relate to a system, wherein, during the insufflation phase of the mechanical cough mode, a flow rate of the fluid conducted to the patient by the respiratory device gradually increases throughout the insufflation phase.

In some aspects, the techniques described herein relate to a system, wherein the controller is configured to issue one or more commands to the respiratory device such that, during the insufflation phase of the mechanical cough mode, the pressure of the fluid conducted to the patient by the respiratory device substantially follows a line having a constant positive slope.

In some aspects, the techniques described herein relate to a system, wherein the controller is configured to issue one or more commands to the respiratory device such that, during the insufflation phase of the mechanical cough mode, the pressure of the fluid conducted to the patient by the respiratory device oscillates relative to the line.

In some aspects, the techniques described herein relate to a system, wherein the controller is configured to issue one or more commands to the respiratory device such that, during the insufflation phase of the mechanical cough mode, the flow rate of the fluid conducted to the patient by the respiratory device substantially follows a line having a constant positive slope.

In some aspects, the techniques described herein relate to a system, wherein the controller is configured to issue one or more commands to the respiratory device such that, during the insufflation phase of the mechanical cough mode, the flow rate of the fluid conducted to the patient by the respiratory device oscillates relative to the line.

In some aspects, the techniques described herein relate to a system, further including: a connection; and a patient interface connected to the connection, wherein the respiratory device is configured to conduct flow to the patient through the patient interface via the connection.

In some aspects, the techniques described herein relate to a system, further including: a pressure sensor; and a flow rate sensor, wherein the controller is configured to interpret signals from the pressure sensor as a pressure of the fluid conducted to the patient by the respiratory device and to interpret signals from the flow rate sensor as a flow rate of the fluid conducted to the patient by the respiratory device.

In some aspects, the techniques described herein relate to a system, wherein the respiratory device is operable in a ventilation mode.

In some aspects, the techniques described herein relate to a system, wherein: the respiratory device is a ventilator, and the controller is configured to issue one or more commands to the ventilator such that the mechanical cough mode is activated periodically.

In some aspects, the techniques described herein relate to a system, wherein the respiratory device is a ventilator or a mechanical insufflation-exsufflation device.

In some aspects, the techniques described herein relate to a method, including: conducting fluid to a patient using a respiratory device operating in a mechanical cough mode such that, during an insufflation phase of the mechanical cough mode, (i) a flow rate of the fluid conducted to the patient by the respiratory device is substantially constant throughout the insufflation phase, or (ii) a flow rate of the fluid conducted to the patient by the respiratory device gradually increases throughout the insufflation phase.

In some aspects, the techniques described herein relate to a method, wherein the pressure of the fluid conducted to the patient by the respiratory device substantially follows a line having a constant positive slope.

In some aspects, the techniques described herein relate to a method, wherein the pressure of the fluid conducted to the patient by the respiratory device oscillates relative to the line.

In some aspects, the techniques described herein relate to a method, wherein

the flow rate of the fluid conducted to the patient by the respiratory device substantially follows a line having a constant positive slope.

In some aspects, the techniques described herein relate to a method, wherein the flow rate of the fluid conducted to the patient by the respiratory device oscillates relative to the line.

In some aspects, the techniques described herein relate to a method, wherein the respiratory device is configured to conduct fluid to the patient through a patient interface via a connection.

In some aspects, the techniques described herein relate to a method, wherein a controller issues one or more commands to the respiratory device in response to signals from a pressure sensor or a flow rate sensor.

In some aspects, the techniques described herein relate to a method, wherein the respiratory device is operable in a ventilation mode.

### BRIEF DESCRIPTION OF THE DRAWINGS

FIG. 1 is a block diagram illustrating an exemplary system that includes a ventilator for use by a human patient.

FIG. 2A is a graph of pressure versus time according to a known control scheme.

FIG. 2B is a graph of flow rate versus time according to the known control scheme.

FIG. 3A is a graph of pressure versus time according to a first control scheme of the present disclosure.

FIG. 3B is a graph of flow rate versus time according to the first control scheme.

FIG. 4A is a graph of pressure versus time according to a second control scheme of the present disclosure.

FIG. 4B is a graph of flow rate versus time according to the second control scheme.

FIG. 5A is a graph of pressure versus time according to a third control scheme of the present disclosure.

FIG. 5B is a graph of flow rate versus time according to the third control scheme.

FIG. 6A is a graph of pressure versus time according to a fourth control scheme of the present disclosure.

FIG. 6B is a graph of flow rate versus time according to the fourth control scheme.

### DETAILED DESCRIPTION

This disclosure relates to control schemes for a system including mechanical cough functionality, and corresponding methods. Among other benefits, this disclosure reduces if not eliminates retrograde displacement of secretions within a patient's airway during mechanical insufflation-exsufflation (MIE), which is referred to herein as mechanical cough, and is sometimes referred to as mechanically assisted cough.

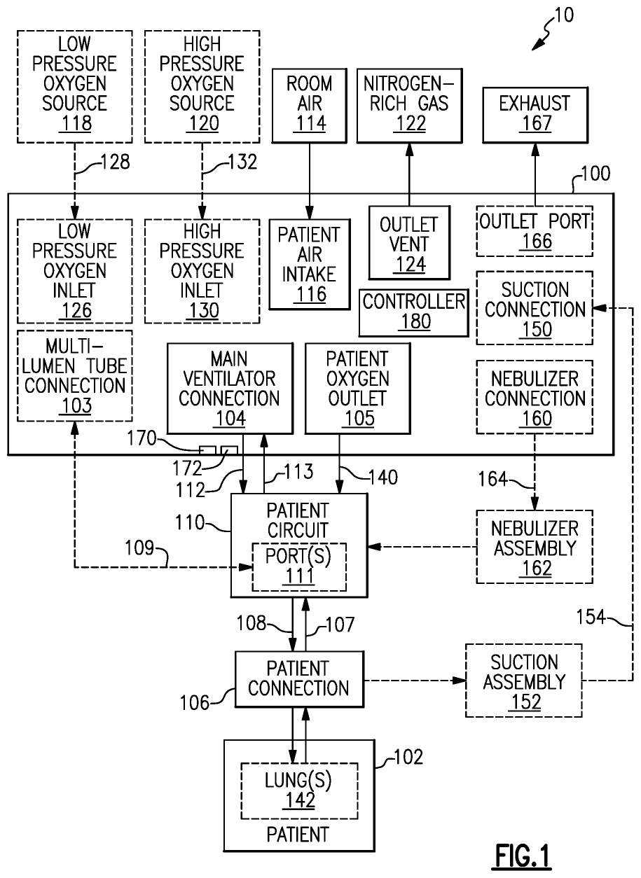

FIG. 1 is a block diagram schematically illustrating an exemplary system 10 that includes a respiratory device 100 with integrated mechanical cough functionality for use by a patient 102, which in an example of this disclosure is a human patient. In FIG. 1, the respiratory device is incorporated into a larger device which also functions as a ventilator. While a ventilator is shown, this disclosure extends to other respiratory devices that are not incorporated into ventilators, including dedicated mechanical insufflation-exsufflation (MIE) devices. Further, while a particular ventilator is described below and shown in FIG. 1, this disclosure extends to other ventilator configurations.

The respiratory device 100 may be configured to provide both traditional volume controlled ventilation and pressure controlled ventilation. The respiratory device 100 has an optional multi-lumen tube connection 103, a main ventilator connection 104, and a patient oxygen outlet 105. The patient 102 has a patient interface, or connection, 106 (e.g., a tracheal tube, a nasal mask, a mouthpiece, and the like) that is connectable to the main ventilator connection 104 and/or the patient oxygen outlet 105 by a patient circuit 110.

As will be described below, the patient circuit 110 may be implemented as an active patient circuit or a passive patient circuit. Optionally, when the patient circuit 110 is implemented as an active patient circuit, the patient circuit 110 may include one or more ports 111 configured to be connected to the optional multi-lumen tube connection 103. The port(s) 111 allow one or more pressure signals 109 to flow between the optional multi-lumen tube connection 103 and the patient circuit 110. A pressure signal may be characterized as gas(es) obtained from a fluid (and/or gas) source for which a pressure is to be measured. The gas(es) obtained are at the same pressure as the fluid (and/or gas) source.

The main respiratory device 100 connection 104 is configured to provide gases 112 that include room air 114 optionally mixed with oxygen. While identified as being “room air,” the room air 114 may include air obtained from any source external to the respiratory device 100. The gases 112 may be used as inspiratory gases (during the inspiratory phase of a breath) or insufflation gases used during the insufflation phase of a cough. The main respiratory device 100 connection 104 is configured to receive gases 113, which may include exsufflation gases exhaled by the patient 102 during an exsufflation phase of a cough.

The air 114 is received by the respiratory device 100 via a patient air intake 116. The oxygen that is optionally mixed with the air 114 may be generated internally by the respiratory device 100 and/or received from an optional low pressure oxygen source 118 (e.g., an oxygen concentrator), and/or an optional high pressure oxygen source 120. When the oxygen is generated internally, the respiratory device 100 may output exhaust gases (e.g., nitrogen-rich gas 122) via an outlet vent 124. Optionally, the respiratory device 100 may include a low pressure oxygen inlet 126 configured to be coupled to the optional low pressure oxygen source 118 and receive optional low pressure oxygen 128 therefrom. The respiratory device 100 may include an optional high pressure oxygen inlet 130 configured to be coupled to the optional high pressure oxygen source 120 and receive optional high pressure oxygen 132 therefrom.

The patient oxygen outlet 105 is configured to provide doses or pulses of oxygen 140 to the patient connection 106 (via the patient circuit 110) that are synchronized with the patient's breathing. Unlike the gases 112 provided by the main respiratory device 100 connection 104, the pulses of oxygen 140 do not include the air 114.

The gases 112 and/or the pulses of oxygen 140 delivered to the patient circuit 110 are conducted thereby as inspiratory or insufflation gases 108 to the patient connection 106, which at least in part conducts those gases into the patient's lung(s) 142. Whenever the patient exhales during the exhalation phase of a breath or exsufflation phase of a cough, exhaled gases 107 enter the patient circuit 110 via the patient connection 106. Thus, the patient circuit 110 may contain one or more of the following gases: the gases 112 provided by the respiratory device 100, the pulses of oxygen 140, and the exhaled gases 107. For ease of illustration, the gases inside the patient circuit 110 will be referred to hereafter as “patient gases.”

Optionally, the respiratory device 100 includes a suction connection 150 configured to be coupled to an optional suction assembly 152. The respiratory device 100 may provide suction 154 to the optional suction assembly 152 via the optional suction connection 150. The suction assembly 152 may be configured to be connected to the patient connection 106, a suction positionable inside the patient connection 106, and/or a drain.

Referring to FIG. 1, optionally, the respiratory device 100 includes a nebulizer connection 160 configured to be coupled to an optional nebulizer assembly 162. The respiratory device 100 may provide gases 164 (e.g., the air 114) to the optional nebulizer assembly 162 via the optional nebulizer connection 160. The optional nebulizer assembly 162 may be configured to be connected to the patient circuit 110.

Optionally, the respiratory device 100 may include an outlet port 166 through which exhaust 167 may exit from the respiratory device 100.

The respiratory device 100 may be configured to be portable and powered by an internal battery (not shown) and/or an external power source (not shown) such as a conventional wall outlet.

The respiratory device 100 further includes a pressure sensor 170 and a flow rate sensor 172. The locations of the pressure and flow rate sensors 170, 172 are exemplary and non-limiting. Further, while both a pressure and flow rate sensor 170, 172 are shown, the respiratory device 100 does not require both a pressure and a flow rate sensor 170, 172, and in one embodiment includes one or the other of the pressure or the flow rate sensor 170, 172.

The pressure and flow rate sensors 170, 172 are configured to generate signals that are interpreted by a controller 180 as a pressure and a flow rate, respectively, of a fluid conducted to the patient 102. While only one of each of the pressure and flow rate sensors 170, 172 are shown in FIG. 1, the respiratory device 100 could include additional pressure and/or flow rate sensors. The controller 180 is configured to follow one or more of a plurality of control schemes and to issue one or more commands to the respiratory device 100, and in particular to one or more components of the respiratory device 100 such as a motor of a compressor and/or one or more valves, to adjust the pressure and/or flow rate of the fluid conducted to the patient 102.

The controller 180 includes a memory connected to one or more processors. The memory is configured to store various tables, algorithms, and instructions, which are executable by the processor(s). The processor(s) may be implemented by one or more microprocessors, microcontroller, application-specific integrated circuits (“ASIC”), digital signal processors (“DSP”), combinations or sub-combinations thereof, or the like. The processor(s) may be integrated into an electrical circuit, such as a conventional circuit board, that supplies power to the processor(s). The processor(s) may include internal memory and/or the memory may be coupled thereto. The present disclosure is not limited by the specific hardware component(s) used to implement the processor(s) and/or the memory.

The memory is a computer readable medium that includes instructions or computer executable components that are executed by the processor(s). The memory may be implemented using transitory and/or non-transitory memory components. The memory may be coupled to the processor(s) by an internal bus.

The memory may include random access memory (“RAM”) and read-only memory (“ROM”). The memory contains instructions and data that control the operation of the processor(s). The memory may also include a basic input/output system (“BIOS”), which contains the basic routines that help transfer information between elements within the respiratory device 100.

Optionally, the memory may include internal and/or external memory devices such as hard disk drives, floppy disk drives, and optical storage devices (e.g., CD-ROM, R/W CD-ROM, DVD, and the like). The respiratory device 100 may also include one or more I/O interfaces (not shown) such as a serial interface (e.g., RS-232, RS-432, and the like), an IEEE-488 interface, a universal serial bus (“USB”) interface, a parallel interface, and the like, for the communication with removable memory devices such as flash memory drives, external floppy disk drives, and the like. In an example, the controller 180 is arranged entirely within the respiratory device 100.

The processor(s) is configured to execute software implementing the processes and control schemes discussed herein, including interpreting information from one or both the sensors 170, 172 and issuing one or more corresponding commands to execute the control schemes discussed herein. Such software may be implemented by the instructions stored in memory.

While a particular embodiment of a respiratory device 100 has been shown in FIG. 1, it should be understood that this disclosure extends to variations of the respiratory device 100, including devices that include lesser or greater functionality relative to the respiratory device 100, and including devices that include fewer or more mechanical components relative to those that are shown in FIG. 1.

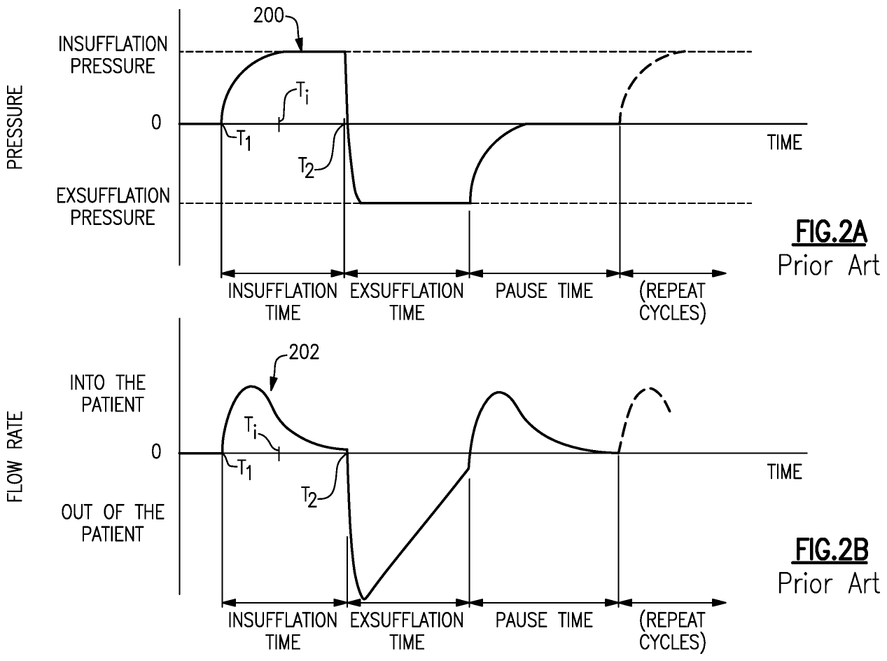

A known control scheme will now be described with reference to FIGS. 2A and 2B. FIGS. 2A and 2B are representative of a device, such as a ventilator or MIE device, conducting fluid to a patient when operating in a mechanical cough mode. In this disclosure, the term “fluid,” when used herein to refer to fluid being conducted relative to the patient by a device, encompasses gas, such as air or patient gases, and any particles or other substances, such as water, medications, or secretions, that may be entrained or suspended in that gas.

FIG. 2A is a graph of a pressure of the fluid conducted to the patient by the device relative to time, and FIG. 2B is a graph of a flow rate of the fluid conducted to the patient by the device relative to time during the same cycle.

The prior art device conducts fluid to a patient following a pressure control line 200 by targeting a set pressure. The flow rate of the fluid conducted to the patient follows line 202. Beginning with the insufflation phase, which occurs during the time labeled insufflation time in the FIGS. 2A and 2B, the prior art device conducts fluid to a patient beginning at time T₁ such that the pressure initially exhibits a relatively steep positive slope. The slope of the control line 200 gradually lessens, while remaining positive, until a set insufflation pressure is reached at an intermediate time Tᵢ. The set insufflation pressure is set by a physician in one example. The pressure is held at the set insufflation pressure between time Tᵢ and T₂, which is when the insufflation phase ends. The result of the steep initial pressure is a relatively high flow rate conducted to the patient between times T₁ and Tᵢ when compared to the flow rates conducted to the patient between times Tᵢ and T₂. The prior art device then completes the cycle by performing an exsufflation phase, pause phase (which is optional), and then, if necessary, repeating the cycle until the secretion has advanced within the airway to a point where it can be removed, either via suction or natural expulsion.

The present disclosure controls the respiratory device 100 in a mechanical cough mode in which the fluid conducted to the patient 102 does not exhibit the relatively steep, abrupt pressure and flow rates at the beginning of the insufflation phase, as in the prior art device of FIGS. 2A and 2B. Rather, in the present disclosure, the controller 180 is configured to issue one or more commands to the respiratory device 100 such that, during an insufflation phase of the mechanical cough mode, a pressure of a fluid conducted to the patient 102 by the respiratory device 100 gradually increases throughout the insufflation phase. Example control schemes will now be described.

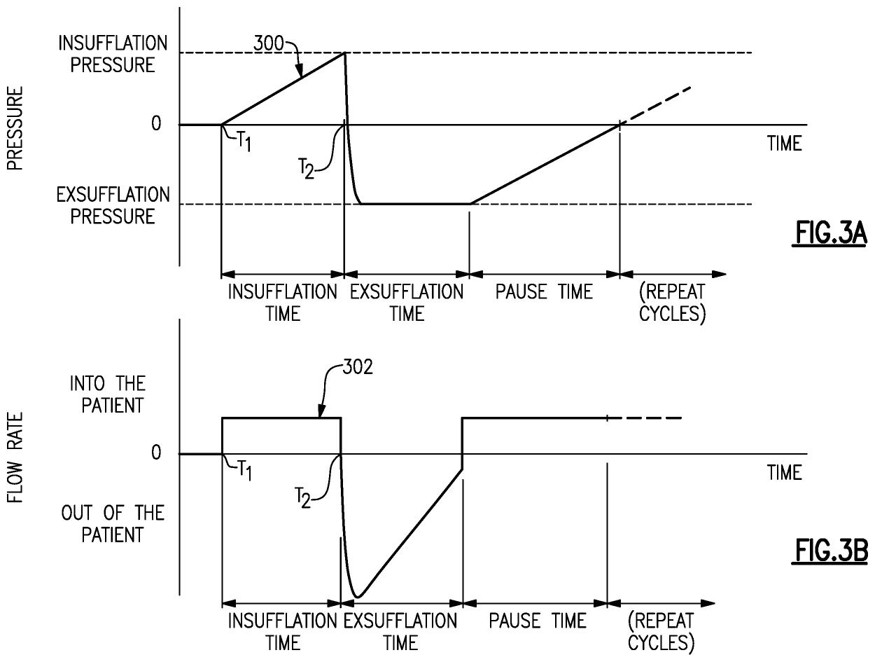

In a first example control scheme, as shown in FIGS. 3A and 3B, the controller 180 is configured to issue one or more commands to the respiratory device 100 such that, during the insufflation phase of the mechanical cough mode, the pressure of the fluid conducted to the patient 102 by the respiratory device 100 substantially follows a control line 300 having a constant positive slope. This approach has the effect of maintaining the flow of the fluid conducted to the patient at essentially the lowest level required to achieve the set pressure by the end of insufflation, and thereby minimizing or eliminating the movement of secretions toward the patient's lungs during the insufflation phase.

In this disclosure, the term “control line” is used to refer to a line representing an active control target of the respiratory device 100, as opposed to, for example, line 302 in FIG. 3B, which is a line representing changes in another flow characteristic as a result of the respiratory device 100 actively following the control line 300. A control line may be embodied as an algorithm and/or lookup table on software of the controller 180. In some embodiments, the control line is a pressure, and in other embodiments the control line is a flow rate. The controller 180 is configured to interpret signals from one or both of the sensors 170, 172 and issue various commands to components of the respiratory device 100 to substantially follow the control line corresponding to a particular control scheme. The term “substantially follow,” when used relative to the controller 180 controlling the respiratory device 100 to substantially follow a control line, is used to refer to the controller 180 actively attempting to follow the control line within acceptable deviations and tolerances in this art.

With continued reference to the embodiment of FIGS. 3A and 3B, in which the control line 300 relates to a pressure, at time T₁ the pressure is zero and at time T₂ the pressure equals the set insufflation pressure. Again, the set insufflation pressure may be set by a physician. Because the control line 300 has a constant positive slope, the pressure conducted to the patient 102 gradually increases at a constant rate throughout the entirety of the insufflation phase. As such, with reference to FIG. 3B, the flow rate, represented by line 302, is substantially constant between times T₁ and T₂ and exhibits a slope of zero. It should be noted that the slope of line 302 is based on a lung compliance of a particular patient, and may not always exhibit a zero slope. Further, in an example, the amplitude of line 302 is less than the flow rate in the prior art device of FIG. 2B. In FIGS. 3A and 3B, the patient 102 does not experience a variable flow rate, including a flow rate that is initially relatively large as shown in FIG. 2B, which reduces if not eliminates the likelihood of retrograde displacement of secretions during the insufflation phase. Retrograde displacement is movement of the secretion within an airway of the patient 102 in the direction toward the lungs 142 of the patient 102, and away from the respiratory device 100. Forward displacement, on the other hand, is movement toward the respiratory device 100. Unless otherwise described, the exsufflation and pause phases (note, again, a pause phase is optional) of this disclosure are controlled substantially similar to how they are controlled in FIGS. 2A and 2B. Cycles are repeated, if necessary, and secretions are either expelled naturally or removed using suction.

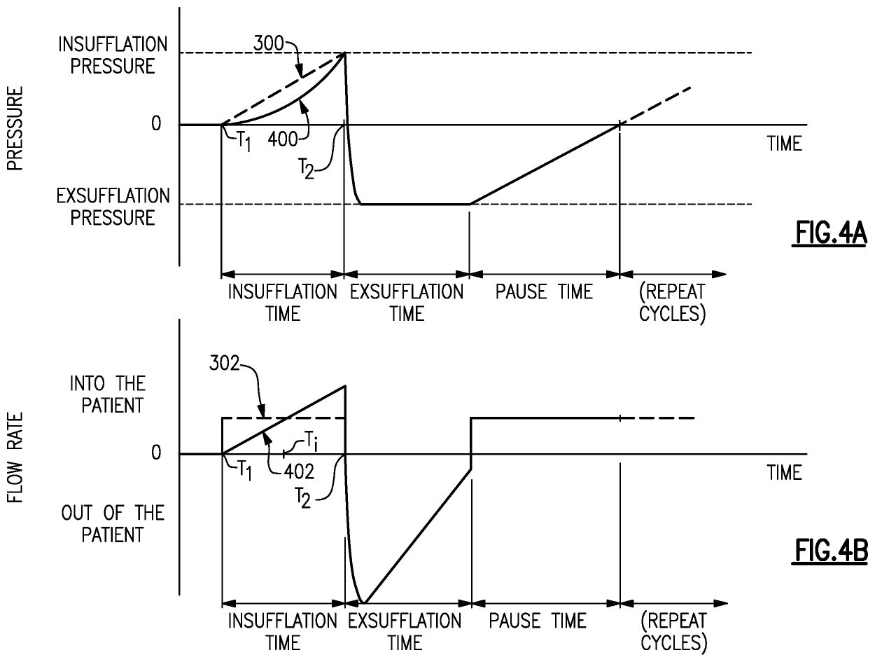

FIGS. 4A and 4B illustrate another example control scheme. In this example, the controller 180 is configured to issue one or more commands to the respiratory device 100 such that, during the insufflation phase of the mechanical cough mode, the flow rate of the fluid conducted to the patient 102 by the respiratory device 100 substantially follows a control line 402 having a constant positive slope. Specifically, unlike in the example of FIGS. 3A and 3B, the respiratory device 100 is controlled to provide a particular flow rate as opposed to a particular pressure. The control line 402 is such that, at time T₁, the flow rate is zero and at time T₂ the flow rate has increased to a point where the pressure, indicated at line 400, equals the set insufflation pressure. Alternatively, the flow rate may increase until the flow rate equals another physician-set value, such as a peak insufflation flow (PIF), which may be within a range between about 10-120 liters per minute (LPM). Because the flow rate gradually increases at a constant rate throughout the entirety of the insufflation phase, the pressure follows a line 400 that gradually increases in steepness, and in particular exhibits a positive slope that gradually increases, between times T₁ and T₂.

Control line 300 and line 302 are shown in FIGS. 4A and 4B for reference. While control line 402 does exceed the flow rate provided in the control scheme of FIGS. 3A and 3B following an intermediate time T₁, the patient 102 never experiences a sharp increase in pressure or flow rate because of the gradually increasing control line 402. Further, the pressure does not exceed a set insufflation pressure, in this example. Thus, the control scheme of FIGS. 4A and 4B also reduces if not eliminates retrograde displacement of secretions.

In FIG. 4B, the control line 402 begins at a flow rate of zero at time T₁. In another example, the control line 402 could begin at a flow rate greater than zero at time T₁. In that example, the control line 402 could remain substantially flat, exhibiting a slope of substantially zero, between times T₁ and T₂. Alternatively, the control line 402 could remain substantially flat while increasing slightly between times Tᵢ and T₂.

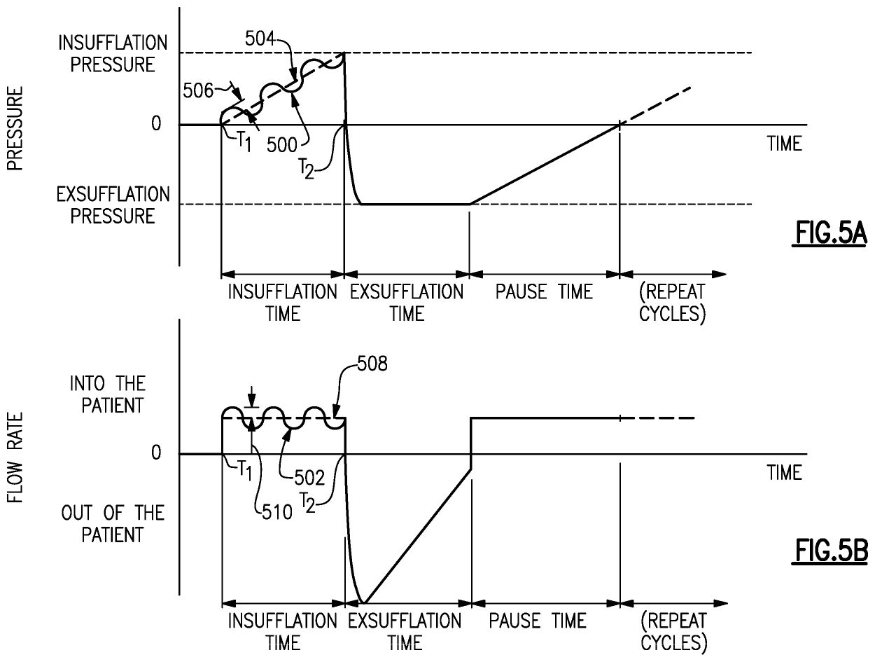

With reference to FIGS. 5A and 5B, another control scheme is disclosed. In this control scheme, during the insufflation phase of the mechanical cough mode, the pressure of the fluid conducted to the patient 102 by the respiratory device 100 substantially follows control line 500, which oscillates about a line 504 having a constant positive slope. In other words, the control line 500 is similar to a sine wave oscillating about an axis, which here is line 504. In this example, line 504 is equivalent to control line 300, and exhibits a pressure of zero at time T₁ and exhibits the set insufflation pressure at time T₂. The control line 500 oscillates by an amplitude 506 relative to the line 504. Beginning at time T₁, the control line 500 oscillates above the line 504. In this example, the control line 500 completes three oscillations relative to line 504 between times T₁ and T₂. This disclosure extends to control lines 500 that complete at least one oscillation between times T₁ and T₂. The resultant flow rate, represented by line 502, conducted to the patient 102 also oscillates relative to line 508 by an amplitude 510. The line 508 is equivalent to line 302 in one example. Oscillating pressure and/or flow rate in this manner may help free secretions within the airway of the patient 102, while also reducing if not eliminating the likelihood of retrograde secretion displacement.

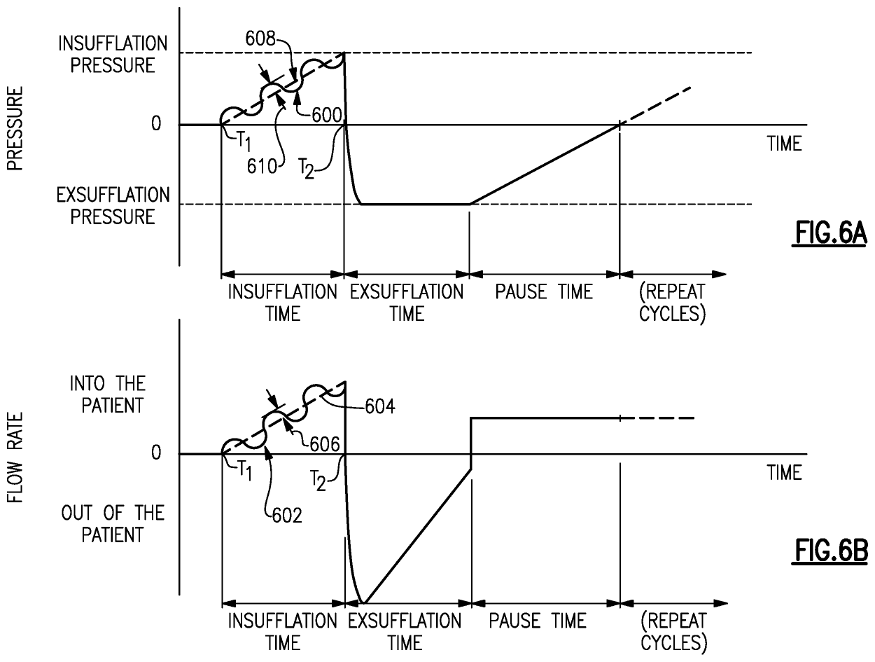

With reference to FIGS. 6A and 6B, still another control scheme is disclosed. In this control scheme, during the insufflation phase of the mechanical cough mode, the flow rate of the fluid conducted to the patient 102 by the respiratory device 100 substantially follows control line 602, which oscillates relative to a line 604 having a constant positive slope. The control line 602 is similar to a sine wave oscillating about an axis, which here is line 604. The line 604 is equivalent to line 402 in one example. The control line 602 oscillates by an amplitude 606 relative to the line 604. Beginning at time T₁, the control line 602 oscillates in a direction above the line 604, and in this example completes three oscillations between times T₁ and T₂. The resultant pressure, represented by line 600, of the fluid conducted to the patient 102 also oscillates relative to line 608 by an amplitude 610. The line 608 exhibits a constant positive slope, in this example. In another example, the line 608 is equivalent to line 400. Again, the oscillations may help free secretions within the airway of the patient 102. In FIGS. 5A, 5B, 6A, and 6B, the oscillations are only present during the insufflation phase, however they could also be present during the exsufflation phase or the pause phase.

In FIGS. 5A and 6B, the control lines 500 and 602 are considered to gradually increase throughout the insufflation phase in this disclosure, despite the oscillations, because the control lines 500, 602 substantially follow, and oscillate about, lines that gradually increase throughout the insufflation phase and because the control lines 500, 602 exhibit minimum values at time T₁ and maximum values at time T₂.

In one aspect of this disclosure, the respiratory device 100 is able to periodically, at pre-programmed intervals, function in a mechanical cough mode. In this aspect, the controller 180 can issue one or more commands to the respiratory device 100 such that the respiratory device 100 delivers fluid according to one of the control schemes of FIGS. 3A-6B at pre-programmed intervals, such as every hour or every four hours, between normal operation of the respiratory device 100 in a ventilation mode. When operating in this mode, the respiratory device 100 may be said to be operating in an “intermittent cough” mode.

It should be understood that terms such as “about,” “substantially,” and “generally” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples. In addition, the various figures accompanying this disclosure are not necessarily to scale, and some features may be exaggerated or minimized to show certain details of a particular component or arrangement.

One of ordinary skill in this art would understand that the above-described embodiments are exemplary and non-limiting. That is, modifications of this disclosure would come within the scope of the claims. Accordingly, the following claims should be studied to determine their true scope and content.

1. A system, comprising:
a respiratory device configured to deliver fluid to a patient, wherein the respiratory device is operable in a mechanical cough mode; and
a controller configured to issue one or more commands to the respiratory device such that, during an insufflation phase of the mechanical cough mode, (i) a flow rate of the fluid conducted to the patient by the respiratory device is substantially constant throughout the insufflation phase, or (ii) a flow rate of the fluid conducted to the patient by the respiratory device gradually increases throughout the insufflation phase.

2. The system as recited in claim 1, wherein, during the insufflation phase of the mechanical cough mode, a flow rate of the fluid conducted to the patient by the respiratory device is substantially constant throughout the insufflation phase.

3. The system as recited in claim 1, wherein, during the insufflation phase of the mechanical cough mode, a flow rate of the fluid conducted to the patient by the respiratory device gradually increases throughout the insufflation phase.

4. The system as recited in claim 1, wherein the controller is configured to issue one or more commands to the respiratory device such that, during the insufflation phase of the mechanical cough mode, the pressure of the fluid conducted to the patient by the respiratory device substantially follows a line having a constant positive slope.

5. The system as recited in claim 4, wherein the controller is configured to issue one or more commands to the respiratory device such that, during the insufflation phase of the mechanical cough mode, the pressure of the fluid conducted to the patient by the respiratory device oscillates relative to the line.

6. The system as recited in claim 1, wherein the controller is configured to issue one or more commands to the respiratory device such that, during the insufflation phase of the mechanical cough mode, the flow rate of the fluid conducted to the patient by the respiratory device substantially follows a line having a constant positive slope.

7. The system as recited in claim 6, wherein the controller is configured to issue one or more commands to the respiratory device such that, during the insufflation phase of the mechanical cough mode, the flow rate of the fluid conducted to the patient by the respiratory device oscillates relative to the line.

8. The system as recited in claim 1, further comprising:
a connection; and
a patient interface connected to the connection, wherein the respiratory device is configured to conduct flow to the patient through the patient interface via the connection.

9. The system as recited in claim 8, further comprising:
a pressure sensor; and
a flow rate sensor, wherein the controller is configured to interpret signals from the pressure sensor as a pressure of the fluid conducted to the patient by the respiratory device and to interpret signals from the flow rate sensor as a flow rate of the fluid conducted to the patient by the respiratory device.

10. The system as recited in claim 1, wherein the respiratory device is operable in a ventilation mode.

11. The system as recited in claim 1, wherein:
the respiratory device is a ventilator, and
the controller is configured to issue one or more commands to the ventilator such that the mechanical cough mode is activated periodically.

12. The system as recited in claim 1, wherein the respiratory device is a ventilator or a mechanical insufflation-exsufflation device.

13. A method, comprising:
conducting fluid to a patient using a respiratory device operating in a mechanical cough mode such that, during an insufflation phase of the mechanical cough mode, (i) a flow rate of the fluid conducted to the patient by the respiratory device is substantially constant throughout the insufflation phase, or (ii) a flow rate of the fluid conducted to the patient by the respiratory device gradually increases throughout the insufflation phase.

14. The method as recited in claim 13, wherein the pressure of the fluid conducted to the patient by the respiratory device substantially follows a line having a constant positive slope.

15. The method as recited in claim 14, wherein the pressure of the fluid conducted to the patient by the respiratory device oscillates relative to the line.

16. The method as recited in claim 13, wherein the flow rate of the fluid conducted to the patient by the respiratory device substantially follows a line having a constant positive slope.

17. The method as recited in claim 16, wherein the flow rate of the fluid conducted to the patient by the respiratory device oscillates relative to the line.

18. The method as recited in claim 13, wherein the respiratory device is configured to conduct fluid to the patient through a patient interface via a connection.

19. The method as recited in claim 18, wherein a controller issues one or more commands to the respiratory device in response to signals from a pressure sensor or a flow rate sensor.

20. The method as recited in claim 13, wherein the respiratory device is operable in a ventilation mode.
